(19) United States
(12) Patent Application Publication
(10) Pub. No.: US 2014/0010876 A1
(43) Pub. Date: Jan. 9, 2014

(54) PHARMACEUTICAL ADMINISTRATION FORMS COMPRISING 5-CHLORO-N-(METHYL)-2-THIOPHENECARBOXAMIDE

(71) Applicant: BAYER PHARMA AKTIENGESELLSCHAFT, Berlin (DE)

(72) Inventors: Klaus BENKE, Bergisch Gladbach (DE); Heike Neumann, Berlin (DE); Wolfgang Mück, Wuppertal (DE)

(73) Assignee: BAYER PHARMA AKTIENGESELLSCHAFT, Berlin (DE)

(21) Appl. No.: 13/930,094

(22) Filed: Jun. 28, 2013

(30) Foreign Application Priority Data
Jul. 3, 2012 (EP) 12174797.6

(51) Int. Cl. A61K 9/00; A61K 31/5377; A61K 9/20

(52) U.S. Cl. CPC A61K 9/0004; A61K 9/2086; A61K 31/5377
USPC 424/472; 424/468; 514/230.8

(57) ABSTRACT

The present invention relates to solid orally administrable pharmaceutical administration forms comprising 5-chloro-N-({(5S)-2-oxo-3-[4-(3-oxo-4-morpholinyl)phenyl]-1,3-oxazolidin-5-yl}-methyl)-2-thiophenecarboxamide (rivaroxaban, active compound (I)), wherein a partial amount of the active compound (I) is released rapidly and a partial amount is released in a controlled manner (modified, retarded, delayed), and to processes for their preparation, their use as medicaments and their use for the prophylaxis, secondary prophylaxis or treatment of disorders.

The present invention relates to solid orally administrable pharmaceutical administration forms comprising 5-chloro-N-({(5S)-2-oxo-3-[4-(3-oxo-4-morpholinyl)phenyl]-1,3-oxazolidin-5-yl}-methyl)-2-thiophenecarboxamide (rivaroxaban, active compound (I)), characterized in that a partial amount of the active compound (I) is released rapidly and a partial amount is released in a controlled manner (modified, retarded, delayed), and to processes for their preparation, their use as medicaments and their use for the prophylaxis, secondary prophylaxis or treatment of disorders.

5-Chloro-N-({(5S)-2-oxo-3-[4-(3-oxo-4-morpholinyl)phenyl]-1,3-oxazolidin-5-yl}methyl)-2-thiophenecarboxamide (rivaroxaban, active compound (I)) is a low-molecular weight orally administrable inhibitor of the blood coagulation factor Xa which can be used for the prophylaxis, secondary prophylaxis and/or treatment of various thromboembolic disorders [WO 01/47919]. If hereinbelow the active compound (I) is referred to, this means the crystal modification I of 5-chloro-N-({(5S)-2-oxo-3-[4-(3-oxo-4-morpholinyl)phenyl]-1,3-oxazolidin-5-yl}methyl)-2-thiophenecarboxamide (I).

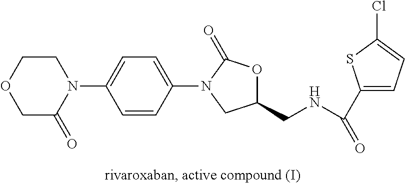 [SMILES: O=C(NC[C@H]1CN(c2ccc(N3CCOCC3=O)cc2)C(=O)O1)c1ccc(Cl)s1]

In the case of diseases which have to be treated for a relatively long period, or for the long-term prophylaxis of diseases, it is desirable to keep the frequency of administration of medicaments as low as possible. This is not only more convenient for the patient but also increases treatment safety by reducing the disadvantages of infrequent administrations (improved compliance). The desired reduction of administration frequency, for example from administration twice daily to administration once daily, can be achieved by extending the therapeutically effective plasma levels via controlled release of active compound from the dosage forms.

Following administration of dosage forms with a controlled release of active compound, as a result in addition of the plasma level being smoothed (minimization of the peak/trough ratio), that is by avoidance of the high active compound plasma concentrations frequently observed after administration of rapid-release drug forms, the occurrence of unwanted side effects correlating with the peak concentrations can be avoided.

In particular for the long-term therapy or prophylaxis and for the secondary prophylaxis of arterial and/or venous thromboembolic disorders (for example deep vein thromboses, stroke, myocardial infarction and pulmonary embolism) it is advantageous to have the active compound (I) available in a form which, via controlled release of active compound, reduces plasma peaks and thus the peak/trough ratio and thus allows administration once daily, with a relatively low food dependency of the pharmacokinetic parameters (in particular cₘₐₓ and AUC) and a relatively high oral bioavailability compared to formulations with rapid release of the active compound (I).

During the development of formulations, the physicochemical characteristics have to be taken into account in combination with the particular biological properties of the active compound (I). The physicochemical characteristics include, for example, the poor solubility of the active compound (I) in water (about 7 mg/l). The particular biological properties of the active compound (I) are a food-dependent bioavailability (the “food effect”) which, in the case of rapid-release tablet formulations, is noticeable at dosages from about 15 mg, and a limited absorption from the lower sections of the intestine. As a consequence, the desired administration once daily requires specific pharmaceutical formulations releasing the active compound (I) taking into account its biopharmaceutical characteristics, such that on the one hand the active compound (I) is present in an amount sufficient to have a pharmaceutical effect and that on the other hand the active compound (I) is released in a manner such that its challenging properties with respect to solubility, food effect and absorption are overcome.

As described in EP 1 830 855 B1, various development approaches were investigated for this purpose, including osmotic two-chamber systems. These were characterized via their in vitro release profile and pharmacokinetic studies with healthy volunteers (n=12) in comparison to a tablet formulation with rapid release of the active compound (I).

The osmotic two-chamber systems prepared according to EP 1 830 855 B1 and described in more detail as comparative formulations A to C in the Experimental Part show the desired smoothing of the blood plasma profiles in comparison to the rapid-release tablet formulations but, surprisingly, have a pronounced food effect. If administration took place after an American breakfast, for example, the maximum blood plasma value measured (c_{max norm}) was increased by a factor of 2.08 to 3.19 in comparison to administration without food (administration on an empty stomach). This relatively pronounced food effect is unusual in particular for osmotic systems. Thus, for example, “Biopharmazie—Pharmakokinetik—Bioverfügbarkeit—Biotransformation” (Langner/Borchert/Mehnert; Wissenschaftliche Verlagsgesellschaft Stuttgart, 4^{th} edition, 2011, page 245) and “Formulation-dependent food effects demonstrated for Nifedipine modified-release preparations marketed in the European Union” (Schug B. S., Brendel E., Wolf D., Wonnemann M., Wargenau M., Blume H. H.; European Journal of Pharmaceutical Sciences, 15, 2002, 279-285) refer to the low food effect of osmotic systems as being a particular advantage.

Accordingly, it was the aim of the development to identify a suitable formulation which, compared to a tablet formulation with rapid release of the active compound (I), has relatively high bioavailability, generates lower plasma level peaks compared to tablet formulations with rapid release of the active compound (I) and allows administration once daily with a relatively small food effect.

Surprisingly, it has now been found that dosage forms releasing part of the active compound dose rapidly and part of the active compound dose at a controlled (modified, retarded) rate allow administration once daily with a relatively small food effect and better oral bioavailability in particular when administered on an empty stomach.

According to the invention, controlled release is to be understood as meaning active compound release characteristics which, following administration, are adjusted, with respect to time, course and/or location in the gastrointestinal tract, in a manner that can not be achieved following administration of conventional formulations such as oral solutions or solid dosage forms with rapid release of the active compound as described, for example, in EP 1 689 370 B1. In addition to the term “controlled release”, use is frequently also made of alternative terms such as “modified”, “retarded” or “delayed” release. These are also embraced by the scope of the present invention.

A pharmaceutical dosage form having combined rapid and controlled release is to be understood as meaning that the active compound dose present in the dosage form is divided into at least two parts, namely into at least one active compound dose which is released rapidly and at least one active compound dose which is released in a controlled manner, which are incorporated into at least two different compartments of the dosage form.

In this manner, 10 to 45% of the active compound (I) (based on the declared total amount of active compound (I)) are released within 1 hour, 40 to 70% of the active compound (I) are released within 4 hours and at least 80% of the active compound (I) are released within 10 hours in accordance with the USP release method with apparatus 2 (paddle) at 50 to 100 rotations per minute (rpm), preferably 75 rpm, in 900 ml of a suitable release medium, preferably a phosphate citrate buffer of pH 6.8, and using a sink in accordance with the Japanese Pharmacopoeia.

The combination of a rapid-release portion of the active compound (IR=immediate release) with a controlled-release portion of the active compound (CR=controlled release) is known in principle. This also applies to osmotic two-chamber systems.

Connor, D. F. and Steingard, R. J., “New Formulations of Stimulants for Attention-Deficit Hyperactivity Disorder”, CNS Drugs 18 (2004), 1011-1030 and Coghill, D., Seth, S., “Osmotic, controlled-release methylphenidate for the treatment of ADHD”, Expert Opinion Pharmacotherapy 7 (2006), 2119-2136 describe various options for combining IR and CR, among others the combination of pellets having different release rates (SODAS™) or the OROS® technology (osmotic systems) combined with an active compound-comprising film coating from which part of the active compound dose is released rapidly. In this case, administration once daily is sought to make up for the very short half-life of the active compound, to reduce side effects and to avoid a second tablet administration during the school time of the children affected. Thus, here the combination with the IR proportion targets the direct onset of activity. In contrast to active compound (I), the substances employed have good solubility.

WO 1993/000071 describes an osmotic system coated with an active compound-comprising film to delay, after an initial active compound release within the first 5 to 15 minutes after administration, the further release of active compound from 30 minutes to 4.5 hours (the target being at least 2 hours). This application is focussed on medicinal problems which have to take into account a circadian rhythm, for example relating to the occurrence of myocardial infarctions whose incidence is highest in the early hours of the morning. Administration of the drug formulation in the evening is supposed to reduce, with the aid of the second burst of active compound after a sufficiently long lag time, the risk of myocardial infarction in the early hours of the morning. Accordingly, the administration includes in particular calcium channel blockers and here especially verapamil hydrochloride. In contrast to active compound (I), the solubility of verapamil hydrochloride in water is very good.

The present invention provides a stable pharmaceutical dosage form which, firstly, comprises a sufficient amount of the active compound (I) for its pharmaceutical effect and which, secondly, releases the active compound (I) in such an amount that the challenging biopharmaceutical properties of the active compound (I) with respect to solubility, food effect and absorption are taken into account. To this end, the active compound (I) is released from the pharmaceutical dosage form both rapidly and in a controlled (modified, retarded) manner, thus allowing, following administration once daily, reduced plasma level peaks in comparison to the rapid-release tablet formulations according to EP 1 689 370 B1, a less strongly pronounced food effect of the pharmacokinetic parameters such as in particular plasma level maxima (cₘₐₓ) and bioavailability (AUC) in comparison to osmotic two-chamber systems without a rapid-release portion according to EP 1 830 855 B1 and moreover a relatively high bioavailability of preferably at least 80% after administration on an empty stomach in comparison to a rapid-release tablet formulation according to EP 1 689 370 B1.

The active compound (I) is present in the crystal modification in which the active compound (I) is obtained by the preparation according to the route described in WO 01/47919 under Example 44 and which is referred to as modification I hereinafter.

In the pharmaceutical dosage forms according to the invention, the active compound (I) is present in crystalline form. In a particularly preferred embodiment of the present invention, the active compound (I) is employed in a micronized form of crystal modification I. Here, the active compound (I) preferably has a mean particle size X₅₀ of less than 10 μm, in particular less than 8 μm, and an X₉₀ value of less than 20 μm, in particular less than 15 μM.

The solid, orally administrable pharmaceutical dosage forms according to the invention comprising 5-chloro-N-({(5S)-2-oxo-3-[4-(3-oxo-4-morpholinyl)phenyl]-1,3-oxazolidin-5-yl}methyl)-2-thiophenecarboxamide (I) are characterized in that they consist of a combination of rapid and controlled release, where
the controlled-release active compound dose is incorporated into an osmotic release system, preferably an osmotic two-chamber system,
and
the osmotic release system is combined with a system that releases the active compound (I) rapidly, preferably an active compound-comprising film coating or an active compound-comprising mantle formed from powder or granules (core/mantle tablet).

The solid, orally administrable pharmaceutical dosage forms according to the invention comprising 5-chloro-N-({5S)-2-oxo-3-[4-(3-oxo-4-morpholinyl)phenyl]-1,3-oxazolidin-5-yl}methyl)-2-thiophenecarboxamide (I) are characterized in that they consist of a combination of rapid and controlled release, where

from the pharmaceutical dosage form

10 to 45% of the active compound (I) of the declared total amount of active compound are released after 1 hour, 40 to 70% of the active compound (I) are released after 4 hours and at least 80% of the active compound (I) are released after 10 hours in accordance with the USP release method using apparatus 2 (paddle).

The solid, orally administrable pharmaceutical dosage forms according to the invention comprising 5-chloro-N-({(5S)-2-oxo-3-[4-(3-oxo-4-morpholinyl)phenyl]-1,3-oxazolidin-5-yl}methyl)-2-thiophenecarboxamide (I) are characterized in that they consist of a combination of rapid and controlled release, where
the controlled-release active compound dose is incorporated into an osmotic release system, preferably an osmotic two-chamber system, and comprises 55 to 90% of the declared total amount of active compound,
the osmotic release system is combined with a system that releases the active compound (I) rapidly, preferably an active compound-comprising film coating or an active compound-comprising mantle formed from powder or granules (core/mantle tablet), and comprises 10 to 45% of the declared total amount of active compound,

and where from the pharmaceutical dosage form
10 to 45% of the active compound (I) of the declared total amount of active compound are released after 1 hour, 40 to 70% of the active compound (I) are released after 4 hours and at least 80% of the active compound (I) are released after 10 hours in accordance with the USP release method using apparatus 2 (paddle).

In the pharmaceutical dosage forms according to the invention, the total dose of the active compound (I) is from 2.5 mg to 30 mg, preferably from 5 mg to 25 mg, particularly preferably 5 mg, 6 mg, 10 mg, 12 mg, 15 mg, 20 mg or 24 mg.

The total amount of active compound is divided into a rapid-release portion of the active compound dose and a controlled-release portion of the active compound does. The rapid-release portion of the active compound dose is 10 to 45% of the active compound (I). The controlled-release portion of the active compound dose is 55 to 90% of the active compound (I).

Suitable dosage forms which allow the active compound (I) to be delivered in a combination of rapid and controlled release are thus based on an osmotic release system in combination with a rapid-release active compound-comprising shell, either in the form of an active compound-comprising film-coating or in the form of a mantle formed from powder or granules (core/mantle tablet) with rapid release of the active compound (I):

1. Controlled Release Using an Osmotic Release System

To realise an osmotic release system, tablets are preferably surrounded with a semipermeable membrane which has at least one opening, preferably one opening. The water-permeable membrane is impermeable for the components of the core, but permits entry of water by osmosis from the outside into the system. Via the resulting osmotic pressure, the water that has penetrated then releases the active compound (I) in dissolved or suspended form from the opening(s) in the membrane. Total active compound release and the release rate can be controlled via thickness and porosity of the semipermeable membrane, the composition of the core and the number and size of the opening(s). Formulation aspects, administration forms and information about preparation processes are described, for example, in the following publications:
Santus, G., Baker, R. W., “Osmotic drug delivery: a review of the patent literature”, Journal of Controlled Release 35 (1995), 1-21
Verma, R. K., Mishra, B., Garg, S., “Osmotically controlled oral drug delivery”, Drug Development and Industrial Pharmacy 26 (2000), 695-708
Verma, R. K., Krishna, D. M., Garg, S., “Formulation aspects in the development of osmotically controlled oral drug delivery systems”, Journal of Controlled Release 79 (2002), 7-27
Verma, R. K., Arora, S., Garg, S., “Osmotic Pumps in drug delivery”, Critical Reviews in Therapeutic Drug Carrier Systems 21 (2004), 477-520
Malaterre, V., Ogorka, J., Loggia, N., Gurny, R. “Oral osmotically driven systems: 30 years of development and clinical use”, European Journal of Pharmaceutics and Biopharmaceutics 73 (2009), 311-323
U.S. Pat. No. 4,327,725, U.S. Pat. No. 4,765,989,

Particularly suitable for the active compound (I) are two-chamber systems (push-pull systems). In the osmotic two-chamber system, the active compound (I) is present in crystalline, preferably micronized form. One advantage of this osmotic two-chamber system in connection with active compound (I) is the uniform release rate which can be set for a relatively long period of time.

In the osmotic two-chamber system, the core consists of two layers: an active compound layer and an osmosis layer. Such an osmotic two-chamber system is described in detail for example in DE 34 17 113 C2.

The active compound layer of the osmotic two-chamber system, which comprises 55 to 90% of the total amount of active compound, is preferably composed of
2 to 25% of active compound (I)
and
60 to 95% of one or more osmotically active polymers, preferably polyethylene oxide of medium viscosity (40 to 100 mPa*s; 5% strength aqueous solution, 25° C.).

The osmosis layer of the osmotic two-chamber system is preferably composed of
40 to 90% of one or more osmotically active polymers, preferably polyethylene oxide of high viscosity (5000 to 8000 mPa*s; 1% strength aqueous solution, 25° C.)
and
10 to 40% of an osmotically active additive.

The difference to 100% in the individual layers of the osmotic two-chamber system (active compound layer and osmosis layer) are independently of one another in each case formed by one or more additional components in the form of pharmaceutically customary auxiliaries. The percentages are in each case based on the total material of the core layer in question.

The active compound layer of the osmotic two-chamber system which comprises 55 to 90% of the total amount of active compound is particularly preferably composed of 5 to 15% of active compound (I).

The active compound layer of the osmotic two-chamber system is particularly preferably composed of 75 to 90% of one or more osmotically active polymers, preferably polyethylene oxide of medium viscosity (40 to 100 mPa*s; 5% strength aqueous solution, 25° C.).

The present invention furthermore provides solid orally administrable pharmaceutical dosage forms, characterized in that they consist of a combination of rapid release and controlled release, where 55 to 90% of the osmotic two-chamber system that comprises the active compound (I) consists of
A) an active compound layer having the composition
2 to 25% of active compound (I),
60 to 95% of one or more osmotically active polymers
B) an osmosis layer having the composition
40 to 90% of one or more osmotically active polymers,
10 to 40% of an osmotically active additive
and
C) a shell consisting of a material which is water-permeable but impermeable for the components of the core, which material has at least one opening on the active compound side.

Suitable for use as osmotically active additives in the core of the osmotic two-chamber system are, for example, all water-soluble substances whose pharmaceutical use is acceptable, such as the water-soluble auxiliaries mentioned in pharmacopoeia or in “Hager” and “Remington Pharmaceutical Science”. Use can be made in particular of water-soluble salts of inorganic or organic acids or non-ionic organic substances having a high solubility in water, such as carbohydrates, in particular sugars, sugar alcohols or amino acids. According to the invention, particular preference is given to using sodium chloride.

Hydrophilic swellable polymers which are optionally additionally present in the core are, for example, hydroxypropylcellulose, hydroxypropylmethylcellulose, sodium carboxymethylcellulose, sodium carboxymethylstarch, polyacrylic acids and their salts.

Pharmaceutically customary auxiliaries which are optionally additionally present in the core are, for example, buffer substances such as sodium bicarbonate, binders such as hydroxypropylcellulose, hydroxypropylmethylcellulose and/or polyvinylpyrrolidone, lubricants such as magnesium stearate, wetting agents such as sodium lauryl sulphate, flow regulators such as finely divided silica and a colour pigment such as iron oxide in one of the two layers for colour differentiation between active compound layer and osmosis layer.

The semipermeable membrane of the osmotic drug release system consists of a material which is water-permeable but impermeable for the components of the core. Such membrane materials are known in principle and described, for example, in EP 1 024 793 B1, pages 3-4, the disclosure of which is hereby included by way of reference. According to the invention, preference is given to using, as membrane material, cellulose acetate or mixtures of cellulose acetate and polyethylene glycol. The semipermeable membrane preferably comprises 5 to 20% pore formers based on the dry weight of the semipermeable membrane. The proportion of the semipermeable membrane (dry weight) in the osmotic dosage form according to the invention is usually 2 to 15%.

The present invention furthermore provides a process for preparing the osmotic two-chamber system according to the invention which comprises mixing and preferably granulating the components of the active compound layer and mixing and preferably granulating the components of the osmosis layer, where dry granulation by roller compacting is preferred for preparing the two layers, and the two types of granules are then compressed on a bilayer tablet press to give a bilayer tablet. The resulting core is then coated with a semipermeable membrane and the shell is provided with one or more openings on the active compound side.

To ensure initial release of active compound, the osmotic release system has to be coated with an active compound-comprising film coating or an active compound-comprising mantle formed from powder or granules. Specifically in the case of osmotic active compound release systems based on cellulose acetate, when a further layer is applied, the best compromise between homogeneous active compound distribution in the resulting coating (film coating or mantle formed from powder or granules), its thickness and process duration or stability has to be found, independently of whether the coating is an active compound-comprising film coating or an active compound-comprising mantle formed from powder or granules.

2. Rapid-Release Active Compound Shell

2.1 Active Compound in the Film Coating

The film-forming polymers used for applying active compound-comprising film coatings can be cellulose derivatives, synthetic polymers and mixtures thereof. Cellulose derivatives which may be mentioned are methylcellulose, hydroxymethylpropylcellulose, hydroxypropylcellulose, carboxymethylcellulose-sodium, hydroxyethylcellulose and mixtures thereof. Synthetic polymers which may be mentioned are polyvinylpyrrolidone, vinylpyrrolidone/vinyl acetate copolymer, polyvinyl alcohol (PVA), polyvinyl acetate, partially hydrolyzed polyvinyl alcohol, polyvinyl alcohol/polyethylene glycol (PEG) copolymers and mixtures thereof. In this case, preferred film-formers are polyvinyl alcohol, polyvinyl acetate, partially hydrolyzed polyvinyl alcohol, polyvinyl alcohol/polyethylene glycol copolymers and mixtures thereof.

The film coating may comprise further auxiliaries such as wetting agents (for example sodium salts of fatty alcohol sulphates such as sodium lauryl sulphate, sulphosuccinates such as sodium dioctylsulphosuccinate, partial fatty esters of polyhydric alcohol such as glycerol monostearate or partial fatty esters of sorbitan such as sorbitan monolaurate), pigments (for example titanium dioxide, talc), colour pigments (for example iron oxide red, yellow or black, or mixtures thereof), release agents (for example kaolin, talc, finely divided silica, magnesium stearate, glycerol monostearate), and/or plasticisers (for example polyethylene glycol, polypropylene glycol, propylene glycol, glycerol, triacetin, triethyl citrate).

Use can also be made of commercially available preparations, “finished coatings”, which already comprise further pharmaceutical auxiliaries and which can simply be dispersed in water. Examples which may be mentioned are:
Kollicoat IR finished coatings (BASF; PVA-co-PEG-based), consisting of Kollicoat IR, Kollidon VA64, kaolin, sodium lauryl sulphate and optionally other colour pigments and
Opadry II coatings (Colorcon; PVA-based), consisting of PVA (partially hydrolyzed), talc, polyethylene glycol, titanium dioxide and optionally iron oxides and lecithin.

In the active compound-comprising film coating, the proportion of the active compound (I) is preferably 5 to 30%, particularly preferably 10 to 25%, and the proportion of wetting agent is preferably 0.1 to 2%, particularly preferably 0.25 to 1%, based on the dry weight of the film coating. The proportion of the film coating in the dosage form according to the invention is preferably in the range of 5 to 15%.

2.2. Powder or Granule Shell as Mantle Coating (Core/Mantle Tablet)

Owing to the unfavourable surface properties of the membrane-coated osmotic release system, the dimensions of the mantle coating (=mantle formed from powder or granules) have to be sufficient to ensure a satisfactory stability of the mantle. The mantle formed from powder or granules which rapidly releases the active compound (I) comprises the customary auxiliaries known to the person skilled in the art, for example wetting agents in the form of surfactants (for example sodium lauryl sulphate, polysorbates), binders (for example sugars, sugar alcohols, starches, cellulose derivatives, alginates, pectines, polyethylene glycols or polyvinylpyrrolidone), fillers in the form of cellulose derivatives (for example microcrystalline cellulose), starches (native or modified, for example potato starch), sugars (for example lactose), sugar alcohols (for example mannitol, sorbitol) and also inorganic fillers (for example calcium phosphate, magnesium oxide), disintegrants in the form of starch derivatives (for example crosslinked sodium carboxymethyl starch, sodium starch glycolate), cellulose derivatives (for example crosslinked carboxymethylcellulose) or crosslinked polyvinylpyrrolidone, and glidants (here as a general term for lubricants/glidants/flow improvers), for example magnesium stearate, calcium stearate, stearic acid, talc and finely divided silica.

The mantle shell according to the invention is prepared by methods known to the person skilled in the art, preferably as described in EP 1 689 370 B1 by fluidized bed granulation. The granules which rapidly release the active compound (I) are then employed as shell granules and, with the osmotic two-chamber system as core, pressed on a core/mantle tablet press to give a core/mantle tablet.

In the mantle coating, the proportion of the active compound (I) is preferably 0.5 to 10%, particularly preferably 1 to 5%, based on the weight of the mantle formed from powder or granules. Up to 1% of a wetting agent may optionally be added, and the proportion of the mantle formed from powder or granules of the total mass of the dosage form according to the invention is preferably at least 50%.

The present invention furthermore provides oral medicaments which are administered once daily and which comprise a dosage form according to the invention.

The present invention furthermore provides a process for preparing the solid orally administrable pharmaceutical dosage form according to the invention having combined rapid and controlled release, characterized in that the components of the active compound layer are mixed and preferably granulated, the components of the osmosis layer are mixed and preferably granulated, and the two granulates are then compressed on a bilayer tablet press to give a bilayer tablet and the resulting core is then coated with a semipermeable membrane and the shell is provided on the active compound side with one or more openings and the resulting membrane-coated core is then surrounded with a rapid-release active compound layer either by applying a film coating comprising the active compound (I) or by pressing active compound-comprising granules prepared by fluidized bed granulation onto the core.

The present invention furthermore provides medicaments comprising a solid orally administrable pharmaceutical dosage form according to the invention which comprises the active compound (I) and releases the active compound (I) in a combined rapid and controlled (modified, retarded) manner.

The present invention furthermore provides the use of a solid orally administrable pharmaceutical dosage form which comprises the active compound (I) and has combined rapid and controlled release for preparing a medicament for the prophylaxis, secondary prophylaxis and/or treatment of disorders, in particular arterial and/or venous thromboembolic disorders.

The present invention furthermore provides the use of the solid orally administrable pharmaceutical dosage form according to the invention with combined rapid and controlled release comprising the active compound (I) for the prophylaxis, secondary prophylaxis and/or treatment of disorders, in particular arterial and/or venous thromboembolic disorders such as myocardial infarction, angina pectoris (including unstable angina), reocclusions and restenoses following angioplasty or aortocoronary bypass, stroke, transitory ischaemic attacks, peripheral arterial occlusion diseases, pulmonary embolisms or deep vein thromboses.

The present invention furthermore provides the use of the solid orally administrable pharmaceutical dosage form according to the invention which comprises the active compound (I) and has combined rapid and controlled release for preparing a medicament for the prophylaxis, secondary prophylaxis and/or treatment of disorders, in particular arterial and/or venous thromboembolic disorders such as myocardial infarction, angina pectoris (including unstable angina), reocclusions and restenoses following angioplasty or aortocoronary bypass, stroke, transitory ischaemic attacks, peripheral arterial occlusion diseases, pulmonary embolisms or deep vein thromboses.

The present invention furthermore provides the use of 5-chloro-N-{(5S)-2-oxo-3-[4-(3-oxo-4-morpholinyl)phenyl]-1,3-oxazolidin-5-yl}methyl)-2-thiophenecarboxamide (I) for preparing a solid orally administrable pharmaceutical dosage form according to the invention with combined rapid and controlled release.

The present invention furthermore provides a method for the prophylaxis, secondary prophylaxis and/or treatment of arterial and/or venous thromboembolic disorders by administering a solid orally administrable pharmaceutical dosage form according to the invention which comprises the active compound (I) and has combined rapid and controlled release.

Below, the invention is illustrated in more detail by preferred embodiments; however, the invention is not limited to these embodiments. Unless indicated otherwise, all amounts given below are in percent by weight.

### EXPERIMENTAL PART

Unless indicated otherwise, the in vitro release studies described below are carried out according to the USP release method using apparatus 2 (paddle). The rotational speed of the stirrer is 75 rpm (rotations per minute) in 900 ml of a phosphate/citrate buffer of pH 6.8 prepared from 1.25 ml of orthophosphoric acid, 4.75 g of citric acid monohydrate and 27.47 g of disodium hydrogen phosphate dihydrate in 10 l of water. To set sink conditions, sodium lauryl sulphate is added to the solution. The amount added depends on the dosage; preferably, the amount added is 0.05 to 0.5% sodium lauryl sulphate, particularly preferably 0.2 to 0.4% sodium lauryl sulphate. Preferably, 0.2% sodium lauryl sulphate is added at an active compound dose of 5 to 10 mg of rivaroxaban, 0.3% sodium lauryl sulphate is added at a dose of 11 to 15 mg of rivaroxaban and 0.4% sodium lauryl sulphate is added at a dose of 16 to 24 mg of rivaroxaban. The person skilled in the art is aware that a minimum amount of sodium lauryl sulphate is required depending on the dose of the active compound to achieve sink conditions. However, the release profiles do not change significantly if the amount of sodium lauryl sulphate is increased above this minimum amount. The release from the tablets takes place from a sinker according to the Japanese Pharmacopoeia.

### Exemplary Formulation 1

### Declared Content=12 Mg/Tablet

Core
active compound layer:

| | | |
|---|---|---|
| active compound (I), micronized | 8.4 | mg |
| hydroxypropylmethylcellulose (5 cp) | 5.7 | mg |
| polyethylene oxide * | 94.8 | mg |
| finely divided silica (Aerosil 200) | 0.9 | mg |
| magnesium stearate | 0.3 | mg |
| | 110.1 | mg |

osmosis layer:

| | | |
|---|---|---|
| hydroxypropylmethylcellulose (5 cp) | 3.69 | mg |
| sodium chloride | 21.51 | mg |
| polyethylene oxide ** | 47.60 | mg |
| iron oxide red | 0.72 | mg |
| magnesium stearate | 0.18 | mg |
| | 73.70 | mg |

Membrane coating

| | | |
|---|---|---|
| cellulose acetate | 17.16 | mg |
| polyethylene glycol 3350 | 2.28 | mg |
| | 19.44 | mg |

Film coating

| | | |
|---|---|---|
| active compound (I), micronized | 4.0 | mg |
| sodium lauryl sulphate | 0.1 | mg |
| Opadry II 85G35294 pink *** | 15.9 | mg |
| | 20.0 | mg |

* viscosity 5% strength aqueous solution (25° C., Brookfield viscosimeter model RVT, spindle No. 1, speed: 50 rpm): 40-100 mPa · s (for example POLYOX ™ Water-Soluble Resin NF WSR N-80; Dow)
** viscosity 1% strength aqueous solution (25° C., Brookfield viscosimeter model RVF, spindle No. 2, speed: 2 rpm): 5000-8000 mPa · s (for example POLYOX ™ Water-Soluble Resin NF WSR Coagulant; Dow)
*** polyvinyl alcohol (partially hydrolyzed), talc, polyethylene oxide 3350, lecithin, titanium dioxide, iron oxide

Preparation:

The components of the active compound layer are mixed and subjected to dry granulation (roller granulation). The components of the osmosis layer are likewise mixed and subjected to dry granulation (roller granulation). On a bilayer tabletting press, the two types of granules are compressed to give a bilayer tablet (diameter 8 mm). The tablets are coated with a solution of cellulose acetate and polyethylene glycol in acetone and dried. Each tablet is then provided on the active compound side with an opening having a diameter of 0.9 mm using a hand drill. For the subsequent coating of the tablets, a suspension is prepared by initially dissolving sodium lauryl sulphate in water, then suspending the active compound (I) in this solution and then dispersing the Opadry finished coating in this suspension.

In Vitro Release of Exemplary Formulation 1

| time [min] | 60 | 120 | 240 | 360 | 480 | 600 |
|---|---|---|---|---|---|---|
| release [%] | 33 | 43 | 64 | 85 | 100 | 102 |

(USP paddle, 75 rpm, 900 ml of phosphate buffer pH 6.8+0.3% NaLS, JP sinker)

### Exemplary Formulation 2

### Declared Content=14 Mg/Tablet

Core
active compound layer:

| | | |
|---|---|---|
| active compound (I), micronized | 8.4 | mg |
| hydroxypropylmethylcellulose (5 cp) | 5.7 | mg |
| polyethylene oxide * | 94.8 | mg |
| finely divided silica (Aerosil 200) | 0.9 | mg |
| magnesium stearate | 0.3 | mg |
| | 110.1 | mg |

osmosis layer:

| | | |
|---|---|---|
| hydroxypropylmethylcellulose (5 cp) | 3.69 | mg |
| sodium chloride | 21.51 | mg |
| polyethylene oxide ** | 47.60 | mg |
| iron oxide red | 0.72 | mg |
| magnesium stearate | 0.18 | mg |
| | 73.70 | mg |

Membrane coating

| | | |
|---|---|---|
| cellulose acetate | 22.5 | mg |
| polyethylene glycol 3350 | 2.25 | mg |
| | 24.75 | mg |

Mantle coating

| | | |
|---|---|---|
| active compound (I), micronized | 6.0 | mg |
| hydroxypropylmethylcellulose, 5 cP | 14.4 | mg |
| sodium lauryl sulphate | 0.9 | mg |
| microcrystalline cellulose | 192.0 | mg |
| lactose monohydrate | 177.4 | mg |
| croscarmellose-sodium | 14.4 | mg |
| magnesium stearate | 2.9 | mg |
| | 408.0 | mg |

* viscosity 5% strength aqueous solution (25° C., Brookfield viscosimeter model RVT, spindle No. 1, speed: 50 rpm): 40-100 mPa · s (for example POLYOX ™ Water-Soluble Resin NF WSR N-80; Dow)
** viscosity 1% strength aqueous solution (25° C., Brookfield viscosimeter model RVF, spindle No. 2, speed: 2 rpm): 5000-8000 mPa · s (for example POLYOX ™ Water-Soluble Resin NF WSR Coagulant; Dow)

Preparation:

Core:

The components of the active compound layer are mixed and subjected to dry granulation (roller granulation). The components of the osmosis layer are likewise mixed and subjected to dry granulation (roller granulation). On a bilayer tabletting press, the two types of granules are compressed to give a bilayer tablet (diameter 8 mm).

Membrane Coating:

The tablets are coated with a solution of cellulose acetate and polyethylene glycol in acetone and dried. Each tablet is then provided on the active compound side with an opening having a diameter of 0.9 mm using a hand drill.

Mantle Coating:

Hydroxypropylmethylcellulose, 5 cP and sodium lauryl sulphate are dissolved in water. The micronized active compound (I) is suspended in this solution. In a fluidized-bed granulation, the suspension prepared in this manner is sprayed as a granulation liquid onto the initial charge of microcrystalline cellulose, lactose monohydrate and croscarmellose sodium. After drying and sieving (mesh size 0.8 mm) of the granules formed, magnesium stearate is added and the components are mixed.

In a core/mantle tablet press, the granules obtained in this manner and the cores already coated with a membrane are compressed to give core/mantle tablets (diameter 12 mm).

In Vitro Release of Exemplary Formulation 2

| time [min] | 60 | 120 | 240 | 360 | 480 | 600 |
|---|---|---|---|---|---|---|
| release [%] | 44 | 47 | 61 | 75 | 88 | 98 |

(USP paddle, 75 rpm, 900 ml of phosphate buffer pH 6.8+0.3% NaLS, JP sinker)

### Exemplary Formulation 3

### Declared Content=12 Mg/Tablet

Core
active compound layer:

| | | |
|---|---|---|
| active compound (I), micronized | 10.0 | mg |
| hydroxypropylmethylcellulose (5 cp) | 5.7 | mg |
| polyethylene oxide * | 93.2 | mg |
| finely divided silica (Aerosil 200) | 0.9 | mg |
| magnesium stearate | 0.3 | mg |
| | 110.1 | mg |

osmosis layer:

| | | |
|---|---|---|
| hydroxypropylmethylcellulose (5 cp) | 3.69 | mg |
| sodium chloride | 21.51 | mg |
| polyethylene oxide ** | 47.60 | mg |
| iron oxide red | 0.72 | mg |
| magnesium stearate | 0.18 | mg |
| | 73.70 | mg |

Membrane coating

| | | |
|---|---|---|
| cellulose acetate | 14.58 | mg |
| polyethylene glycol 3350 | 1.94 | mg |
| | 16.52 | mg |

Film coating

| | | |
|---|---|---|
| active compound (I), micronized | 2.5 | mg |
| sodium lauryl sulphate | 0.1 | mg |
| Opadry II 85G35294 pink *** | 17.4 | mg |
| | 20.0 | mg |

* viscosity 5% strength aqueous solution (25° C., Brookfield viscosimeter model RVT, spindle No. 1, speed: 50 rpm): 40-100 mPa · s (for example POLYOX ™ Water-Soluble Resin NF WSR N-80; Dow)
** viscosity 1% strength aqueous solution (25° C., Brookfield viscosimeter model RVF, spindle No. 2, speed: 2 rpm): 5000-8000 mPa · s (for example POLYOX ™ Water-Soluble Resin NF WSR Coagulant; Dow)
*** polyvinyl alcohol (partially hydrolyzed), talc, polyethylene oxide 3350, lecithin, titanium dioxide, iron oxide

The preparation was carried out analogously to Exemplary Formulation 1.

In Vitro Release of Exemplary Formulation 3

| time [min] | 60 | 120 | 240 | 360 | 480 | 600 |
|---|---|---|---|---|---|---|
| release [%] | 19 | 32 | 59 | 85 | 96 | 96 |

(USP paddle, 75 rpm, 900 ml of phosphate buffer pH 6.8+0.3% NaLS, JP sinker)

### Exemplary Formulation 4

### Declared Content=12 Mg/Tablet

Core
active compound layer:

| | | |
|---|---|---|
| active compound (I), micronized | 10.0 | mg |
| hydroxypropylmethylcellulose (5 cp) | 5.7 | mg |
| polyethylene oxide * | 93.2 | mg |
| finely divided silica (Aerosil 200) | 0.9 | mg |
| magnesium stearate | 0.3 | mg |
| | 110.1 | mg |

osmosis layer:

| | | |
|---|---|---|
| hydroxypropylmethylcellulose (5 cp) | 3.69 | mg |
| sodium chloride | 21.51 | mg |
| polyethylene oxide ** | 47.60 | mg |
| iron oxide red | 0.72 | mg |
| magnesium stearate | 0.18 | mg |
| | 73.70 | mg |

Membrane coating

| | | |
|---|---|---|
| cellulose acetate | 17.16 | mg |
| polyethylene glycol 3350 | 2.28 | mg |
| | 19.44 | mg |

Film coating

| | | |
|---|---|---|
| active compound (I), micronized | 2.5 | mg |
| sodium lauryl sulphate | 0.1 | mg |
| Opadry II 85G35294 pink *** | 17.4 | mg |
| | 20.0 | mg |

* viscosity 5% strength aqueous solution (25° C., Brookfield viscosimeter model RVT, spindle No. 1, speed: 50 rpm): 40-100 mPa · s (for example POLYOX ™ Water-Soluble Resin NF WSR N-80; Dow)
** viscosity 1% strength aqueous solution (25° C., Brookfield viscosimeter model RVF, spindle No. 2, speed: 2 rpm): 5000-8000 mPa · s (for example POLYOX ™ Water-Soluble Resin NF WSR Coagulant; Dow)
*** polyvinyl alcohol (partially hydrolyzed), talc, polyethylene oxide 3350, lecithin, titanium dioxide, iron oxide

The preparation was carried out analogously to Exemplary Formulation 1.

In Vitro Release of Exemplary Formulation 4

| time [min] | 60 | 120 | 240 | 360 | 480 | 600 |
|---|---|---|---|---|---|---|
| release [%] | 19 | 27 | 50 | 72 | 91 | 96 |

(USP paddle, 75 rpm, 900 ml of phosphate buffer pH 6.8+0.3% NaLS, JP sinker)

### Exemplary Formulation 5

### Declared Content=12 Mg/Tablet

Core
active compound layer:

| | | |
|---|---|---|
| active compound (I), micronized | 10.0 | mg |
| hydroxypropylmethylcellulose (5 cp) | 5.7 | mg |
| polyethylene oxide * | 93.2 | mg |
| finely divided silica (Aerosil 200) | 0.9 | mg |
| magnesium stearate | 0.3 | mg |
| | 110.1 | mg |

osmosis layer:

| | | |
|---|---|---|
| hydroxypropylmethylcellulose (5 cp) | 3.69 | mg |
| sodium chloride | 21.51 | mg |
| polyethylene oxide ** | 47.60 | mg |
| iron oxide red | 0.72 | mg |
| magnesium stearate | 0.18 | mg |
| | 73.70 | mg |

Membrane coating

| | | |
|---|---|---|
| cellulose acetate | 19.73 | mg |
| polyethylene glycol 3350 | 2.62 | mg |
| | 22.35 | mg |

Film coating

| | | |
|---|---|---|
| active compound (I), micronized | 2.5 | mg |
| sodium lauryl sulphate | 0.1 | mg |
| Opadry II 85G35294 pink *** | 17.4 | mg |
| | 20.0 | mg |

* viscosity 5% strength aqueous solution (25° C., Brookfield viscosimeter model RVT, spindle No. 1, speed: 50 rpm): 40-100 mPa · s (for example POLYOX ™ Water-Soluble Resin NF WSR N-80; Dow)
** viscosity 1% strength aqueous solution (25° C., Brookfield viscosimeter model RVF, spindle No. 2, speed: 2 rpm): 5000-8000 mPa · s (for example POLYOX ™ Water-Soluble Resin NF WSR Coagulant; Dow)
*** polyvinyl alcohol (partially hydrolyzed), talc, polyethylene oxide 3350, lecithin, titanium dioxide, iron oxide

The preparation was carried out analogously to Exemplary Formulation 1.

In Vitro Release of Exemplary Formulation 5

| time [min] | 60 | 120 | 240 | 360 | 480 | 600 |
|---|---|---|---|---|---|---|
| release [%] | 18 | 23 | 42 | 61 | 80 | 93 |

(USP paddle, 75 rpm, 900 ml of phosphate buffer pH 6.8+0.3% NaLS, JP sinker)

In Vivo Results: Human Pharmacokinetic Studies

Exemplary Formulation 1 according to the invention was tested in a pharmacokinetic study in healthy volunteers (n=11) crossover in comparison with a tablet formulation with rapid release of the active compound (I) (the formulation is described as Example 5.1/Tablet B in EP 1 689 370 B1), where Exemplary Formulation 1 was administered either on an empty stomach or after an American breakfast to check the food effect of the bioavailability (AUC) and the plasma level peaks (cₘₐₓ values). AUCₙₒᵣₘ and C_{max norm} ratios (ratio Exemplary Formulation 1 (CR=controlled release) to rapid-release tablet formulation (IR=immediate release)) in each case on an empty stomach (fasted) and after an American breakfast (fed) and also the ratio of these values are stated as relevant pharmacokinetic parameters in Table 1.

TABLE 1
Exemplary Formulation 1 in comparison
to a rapid-release tablet 10 mg

| AUC norm ratio | |
|---|---|
| CR fasted/IR fasted | 0.870 |
| CR fed/IR fasted | 1.015 |
| CR fed/CR fasted | 1.17 |
| Cmax norm ratio | |
| CR fasted/IR fasted | 0.582 |
| CR fed/IR fasted | 0.879 |
| CR fed/CR fasted | 1.51 |

CR = controlled release = Exemplary Formulation 1; IR = immediate release tablet

For comparison, the results from pharmacokinetic studies carried out in an analogous manner with Comparative Formulations A to C are listed in a table. These osmotic two-chamber systems without any rapid-release proportion of active compound have the following composition (stated in mg/tablet):

| Comparative formulation | A | B | C |
|---|---|---|---|
| Declared content (mg/tablet) | 10 | 20 | 12 |

Core
Active compound layer

| | | | |
|---|---|---|---|
| active compound (I), micronized | 11.0 | 22.0 | 12.5 |
| hydroxypropylmethylcellulose (5 cP) | 9.1 | 8.5 | 5.7 |
| polyethylene oxide * | 138.1 | 127.7 | 90.7 |
| finely divided silica (Aerosil 200, Degussa) | 1.3 | 1.3 | 0.9 |
| magnesium stearate | 0.6 | 0.6 | 0.3 |
| | 160.1 | 160.1 | 110.1 |

Osmosis layer

| | | | |
|---|---|---|---|
| hydroxypropylmethylcellulose (5 cP) | 4.1 | 4.1 | 3.69 |
| sodium chloride | 23.9 | 23.9 | 21.51 |
| polyethylene oxide ** | 52.9 | 52.9 | 47.6 |
| iron oxide red | 0.8 | 0.8 | 0.72 |
| magnesium stearate | 0.2 | 0.2 | 0.18 |
| | 81.9 | 81.9 | 73.7 |

Osmotic membrane

| | | | |
|---|---|---|---|
| cellulose acetate | 18.0 | 18.0 | 14.3 |
| polyethylene glycol 400 | 3.0 | 3.0 | — |
| polyethylene glycol 3350 | — | — | 1.9 |
| | 21.0 | 21.0 | 16.2 |
| Non-functional colour coating | 6.0 | 6.0 | — |

* viscosity 5% strength aqueous solution (25° C., Brookfield viscosimeter model RVT, spindle No. 1, speed: 50 rpm): 40-100 mPa · s (for example POLYOX ™ Water-Soluble Resin NF WSR N-80; Dow)
** viscosity 1% strength aqueous solution (25° C., Brookfield viscosimeter model RVF, spindle No. 2, speed: 2 rpm): 5000-8000 mPa · s (for example POLYOX ™ Water-Soluble Resin NF WSR Coagulant; Dow)

Tablets A to C were prepared analogously to Exemplary Formulation 3.2 from EP 1 830 855 B1. For Comparative Formulations A and B, the chosen tablet format was 8.7 mm; Comparative Formulation C was compressed to 8 mm tablets.

In vitro release rates [%] of comparative formulations A to C:

| Time [min] | 60 | 120 | 240 | 360 | 480 |
|---|---|---|---|---|---|
| Formulation A | 4 | 20 | 56 | 86 | 101 |
| Formulation B | 4 | 21 | 58 | 89 | 102 |
| Formulation C | 1 | 16 | 52 | 87 | 99 |

(USPTpaddle, 75 rpm, 900 ml of phosphate buffer pH 6.8+NaLS, JP sinker)

Table 2 lists the relevant pharmacokinetic parameters of the AUCₙₒᵣₘ and C_{max norm} ratios in each case for administration on an empty stomach (fasted) and after an American breakfast (fed) (ratio comparative formulations A, B and C(CR) to the rapid-release tablet formulation (IR) and also the ratio of these values).

TABLE 2
Comparative Formulations A to C in comparison
to rapid-release tablets 10 mg

| | A | B | C |
|---|---|---|---|
| AUC norm ratio | | | |
| CR fasted/IR fasted | 0.730 | 0.669 | 0.615 |
| CR fed/IR fasted | 0.961 | 0.944 | 0.989 |
| CR fed/CR fasted | 1.32 | 1.41 | 1.61 |
| Cmax norm ratio | | | |
| CR fasted/IR fasted | 0.265 | 0.354 | 0.220 |
| CR fed/IR fasted | 0.646 | 0.736 | 0.702 |
| CR fed/CR fasted | 2.41 | 2.08 | 3.19 |

CR = controlled release = Comparative Formulations A to C; IR = immediate release tablet

This demonstrates the superiority of Exemplary Formulation 1 in comparison to Formulations A to C. Whereas the Comparative Formulations A to C have c_{max norm} factors fed/fasted (that is administration after an American breakfast in comparison to administration on an empty stomach) of from 2.08 to 3.19, for Exemplary Formulation 1 this value is only 1.51—thus, the food effect of the c_{max norm} values is markedly less pronounced.

The food effect of the relative bioavailability for Exemplary Formulation 1 is likewise less pronounced (factor 1.17 in comparison to 1.32 to 1.61 for Comparative Formulations A to C). It was furthermore possible to increase the relative bioavailability following administration on an empty stomach for Exemplary Formulation 1 to 87% in comparison to 61.5 to 73% for Comparative Formulations A to C.

1. A solid orally administrable pharmaceutical dosage form comprising 5-chloro-N-({(5S)-2-oxo-3-[4-(3-oxo-4-morpholinyl)phenyl]-1,3-oxazolidin-5-yl}methyl)-2-thiophenecarboxamide (I), characterized in that it consists of a combination of rapid and controlled release, where the active compound dose with controlled release is incorporated into an osmotic two-chamber system,
and
the osmotic release system is combined with an active compound-comprising film coating with rapid release of active compound (I) or an active compound-comprising mantle formed from powder or granules (core/mantle tablet).

2. The solid orally administrable pharmaceutical dosage form according to claim 1, wherein 55 to 90% of active compound (I) are incorporated as controlled-release portion of the active compound dose into the osmotic two-chamber system and 10 to 45% of active compound (I) are incorporated as rapid-release portion of the active compound dose into the rapid-release film coating or an active-compound comprising mantle formed from powder or granules (core/mantle tablet).

3. The solid orally administrable pharmaceutical dosage form according to claim 1, wherein 55 to 90% of the osmotic two-chamber system that comprises the active compound (I) consists of:
A) an active compound layer having the composition
2 to 25% of active compound (I),
60 to 95% of one or more osmotically active polymers
B) an osmosis layer having the composition
40 to 90% of one or more osmotically active polymers,
10 to 40% of an osmotically active additive
and
C) a shell consisting of a material which is water-permeable but impermeable for the components of the core, which material has at least one opening on the active compound side.

4. The solid orally administrable pharmaceutical dosage form according to claim 1, wherein the active compound-comprising film coating comprising 10 to 45% of active compound (I) is composed of 5 to 30% of active compound (I) and 0.1 to 2% of wetting agent, based on the dry weight of the film coatings.

5. The solid orally administrable pharmaceutical dosage form according to claim 1, wherein the mantle formed from powder or granules (core/mantle tablet) and comprising 10 to 45% of active compound (I) is composed of 0.5 to 10% of active compound (I), based on the weight of the mantle formed from powder or granules.

6. The solid orally administrable pharmaceutical dosage form according to claim 1, wherein the pharmaceutical dosage form releases 10 to 45% of active compound (I) of the declared total amount of active compound within 1 hour, 40 to 70% of active compound (I) within 4 hours and at least 80% of active compound (I) within 10 hours according to USP release method using apparatus 2 (paddle).

7. The solid orally administrable pharmaceutical dosage form according to claim 6, wherein the USP-release method is carried out using apparatus 2 (paddle) at 75 rpm in 900 ml of a phosphate/citrate buffer of pH 6.8 with addition of 0.4% sodium lauryl sulphate as release medium and using a sinker according to the Japanese Pharmacopoeia.

8. The solid orally administrable pharmaceutical dosage form according to claim 1, wherein the total dose of active compound (I) is 2.5 mg to 30 mg.

9. The solid orally administrable pharmaceutical dosage form according to claim 1, wherein the active compound (I) is present in crystalline form.

10. The solid orally administrable pharmaceutical dosage form according to claim 9, wherein the active compound (I) is present in micronized form.

11. The solid orally administrable pharmaceutical dosage form according to claim 1, wherein
the active compound layer of the osmotic two-chamber system comprises polyethylene oxide having a viscosity of 40 to 100 mPa*s (5% strength aqueous solution, 25° C.) as osmotically active polymer and the osmosis layer of the osmotic two-chamber system comprises polyethylene oxide having a viscosity of 5000 to 8000 mPa*s (1% strength aqueous solution, 25° C.) as osmotically active polymer.

12. The solid orally administrable pharmaceutical dosage form according to claim 1, wherein the membrane shell of the osmotic two-chamber system consists of cellulose acetate or a mixture of cellulose acetate and polyethylene glycol.

13. A process for preparing the solid orally administrable pharmaceutical dosage form according to claim 1, wherein the components of the active compound layer are mixed and preferably granulated, the components of the osmosis layer are mixed and preferably granulated, and the two granulates are then compressed on a bilayer tablet press to give a bilayer tablet and the resulting core is then coated with a semipermeable membrane and the shell is provided on the active compound side with one or more openings and the resulting membrane-coated core is then surrounded with a rapid-release active compound layer either by applying a film coating comprising the active compound (I) or by pressing active compound-comprising granules prepared by fluidized bed granulation onto the core.

14. A medicament comprising the solid orally administrable pharmaceutical dosage form according to claim 1.

15. A method for the prophylaxis, secondary prophylaxis and/or treatment of thromboembolic disorders, comprising administering a therapeutically effective amount of the solid orally administrable pharmaceutical dosage form according to claim 1 to a patient in need thereof.
